(19) United States
(12) Patent Application Publication
(10) Pub. No.: US 2025/0241788 A1
(43) Pub. Date: Jul. 31, 2025

(54) SYSTEMS FOR COOLED ENDOCARDIAL INJECTION

(71) Applicant: Boston Scientific Scimed, Inc., Maple Grove, MN (US)

(72) Inventors: Aiden Flanagan, Galway (IE); Tim O'Connor, Galway (IE); Martin Lawrence Fawdry, Galway (IE); Michael Hughes, Galway (IE); Jonathan Heelan, Limerick (IE); Michael James Kelly, Galway (IE); Martyn G. Folan, Galway (IE)

(73) Assignee: Boston Scientific Scimed, Inc., Maple Grove, MN (US)

(21) Appl. No.: 19/036,171

(22) Filed: Jan. 24, 2025

Related U.S. Application Data
(60) Provisional application No. 63/625,587, filed on Jan. 26, 2024.

(51) Int. Cl. A61F 7/12; A61F 7/00; A61M 25/00

(52) U.S. Cl. CPC A61F 7/12; A61F 2007/0056; A61F 2007/126; A61M 2025/0037; A61M 2025/0092; A61M 2025/0095

(57) ABSTRACT

System for cooled endocardial injection are disclosed. An example system may include a steerable catheter having a first lumen therein. The system may include a cooling circuit disposed along at least a portion of the steerable catheter, the cooling circuit having a fluid inlet, a fluid outlet, a proximal end, and a distal end. The cooling circuit may include a first lumen section extending distally along the steerable catheter and including the fluid inlet and a second lumen section extending proximally along the steerable catheter and including the fluid outlet. The system may include an injection catheter disposed in the first lumen. The injection catheter may be configured to deliver a cooled therapeutic fluid to endocardial tissue.

### CROSS-REFERENCE TO RELATED APPLICATIONS

This application claims the benefit of priority under 35 U.S.C. § 119 of U.S. Provisional Application No. 63/625,587, filed Jan. 26, 2024, the entire disclosure of which is hereby incorporated by reference.

### TECHNICAL FIELD

The present disclosure pertains to medical devices, and methods for manufacturing medical devices. More particularly, the present disclosure pertains to systems for cooled endocardial injection.

### BACKGROUND

A wide variety of medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices.

In some aspects, a system for cooled endocardial injection is described. The system comprising: a steerable catheter having a first lumen therein; a cooling circuit disposed along at least a portion of the steerable catheter, the cooling circuit having a fluid inlet, a fluid outlet, a proximal end, and a distal end, the cooling circuit including: a first lumen section extending distally along the steerable catheter and including the fluid inlet; and a second lumen section extending proximally along the steerable catheter and including the fluid outlet; and an injection catheter disposed in the first lumen, wherein the injection catheter is configured to deliver a cooled therapeutic fluid to endocardial tissue.

In some embodiments, which may be used in conjunction with any of the above aspects and embodiments, a longitudinal axis of the first lumen section and a longitudinal axis of the second lumen section are parallel.

In some embodiments, which may be used in conjunction with any of the above aspects and embodiments, the cooling circuit is disposed in a wall of the injection catheter.

In some embodiments, which may be used in conjunction with any of the above aspects and embodiments, the cooling circuit is formed of a metal, a polymer, or a combination thereof.

In some embodiments, which may be used in conjunction with any of the above aspects and embodiments, the cooling circuit is a closed cooling circuit.

In some embodiments, which may be used in conjunction with any of the above aspects and embodiments, the closed cooling circuit includes a cooling fluid disposed therein, and wherein the cooling fluid comprises cooled water, cooled glycerol, liquid nitrogen, liquid carbon dioxide, or any combination thereof.

In some embodiments, which may be used in conjunction with any of the above aspects and embodiments, the first lumen section is a first helical lumen section; and the second lumen section is a second helical lumen section.

In some embodiments, which may be used in conjunction with any of the above aspects and embodiments, adjacent helical elements in each of the first helical lumen section and the second helical lumen section are spaced apart axially.

In some embodiments, which may be used in conjunction with any of the above aspects and embodiments, the first lumen section and the second lumen section together form a second lumen that extends between the fluid inlet and the fluid outlet.

In some embodiments, which may be used in conjunction with any of the above aspects and embodiments, the first helical lumen section is configured in a first helical direction and the second helical lumen section is configured in a second helical direction.

In some embodiments, which may be used in conjunction with any of the above aspects and embodiments, at least a distal end of the first helical lumen section and at least a distal end of the second helical lumen section are formed of shape memory material that is configured to move radially between an unexpanded configuration and an expanded configuration.

In some embodiments, which may be used in conjunction with any of the above aspects and embodiments, the first lumen section is a first elongate lumen section; and the second lumen section is a second elongate lumen section.

In some embodiments, which may be used in conjunction with any of the above aspects and embodiments, the first elongate lumen section and the second elongate lumen section are included in a plurality of elongate lumen sections that are spaced axially along the steerable catheter.

In some embodiments, which may be used in conjunction with any of the above aspects and embodiments, the plurality of elongate lumen sections are equally spaced axially along the steerable catheter.

In some embodiments, which may be used in conjunction with any of the above aspects and embodiments, an inner surface of a wall of the steerable catheter is in direct contact with an outer surface of a wall of the injection catheter.

In some aspects, a system for cooled endocardial injection is described. The system comprising: a steerable catheter assembly including a steerable catheter having wall with a first lumen therein; a closed cooling circuit disposed within the wall of the steerable catheter, the closed cooling circuit having a fluid inlet and a fluid outlet each configured to couple to a fluid circulation device, a proximal end, a distal end, the closed cooling circuit including: a first helical lumen section extending distally along the first lumen and including the fluid inlet; and a second helical lumen section extending proximally along the first lumen and including fluid outlet, wherein the first helical lumen section and the second helical lumen section together define a second lumen that extends from the fluid inlet to the fluid outlet; and an injection catheter disposed within the steerable catheter assembly, the injection catheter including a third lumen and being configured to deliver a cooled therapeutic fluid via the third lumen to endocardial tissue.

In some embodiments, which may be used in conjunction with any of the above aspects and embodiments, the cooled therapeutic fluid is a thermogel.

In some aspects, a system for cooled endocardial injection is described. The system comprising: a steerable catheter assembly including a steerable catheter having wall with a first lumen therein; a closed cooling circuit disposed within the wall of the steerable catheter, the closed cooling circuit having a fluid inlet and a fluid outlet each configured to couple to a fluid circulation device, a proximal end, a distal end, the closed cooling circuit including: a plurality of elongate lumen sections including a first elongate lumen section and a second elongate lumen section, wherein the first elongate lumen section and the second elongate lumen section together form a second lumen that extends between the fluid inlet and the fluid outlet; and an injection catheter disposed within the steerable catheter assembly, the injection catheter including a third lumen and being configured to deliver a cooled therapeutic fluid via the third lumen to endocardial tissue.

In some embodiments, which may be used in conjunction with any of the above aspects and embodiments, the first elongate lumen section is adjacent to the second elongate lumen section.

In some embodiments, which may be used in conjunction with any of the above aspects and embodiments, the system further comprises a manifold configured to fluidically couple a proximal end of the first elongate lumen section to the proximal end of the second elongate lumen section.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a plan overview of an example system for cooled endocardial injection.

FIG. 2 is a schematic depiction of an example steerable catheter for cooled endocardial injection.

FIG. 3 is a schematic depiction of an example steerable catheter for cooled endocardial injection.

FIG. 4 is a plan view of a portion of an example system for cooled endocardial injection.

FIG. 5 is a side view of a portion of an example system for cooled endocardial injection.

FIG. 6 is a plan view of a portion of an example system for cooled endocardial injection.

FIG. 7 is a side view of a portion of an example system for cooled endocardial injection.

FIG. 8 is a schematic representation of a portion of an example system for cooled endocardial injection.

FIG. 9A is a view of an example of a double helix cooling circuit.

FIG. 9B is a view of another example of the double helix cooling circuit.

FIG. 9C is a section view of the example of the double helix cooling circuit.

FIG. 9D is a view of an example of a steerable catheter including a shape memory material in a first configuration.

FIG. 9E is a view of an example of a steerable catheter including a shape memory material in a second configuration.

FIG. 10A is a view of an example elongate cooling circuit.

FIG. 10B is a view of another example elongate cooling circuit.

FIG. 10C is a view of another example of a portion of an elongate cooling circuit.

FIG. 11 is a schematic view of a system including an elongate cooling circuit and a manifold.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure. Further, as used herein, the terms “approximately” and “substantially” indicate a range of values within +/−10% of a stated or implied value. Additionally, terms that indicate the geometric shape of a component/surface refer to exact and approximate shapes.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

A therapeutic gel may be used to help reduce and/or prevent heart failure after a myocardial infarction (MI). For example, a clinician may inject a therapeutic gel into the endocardium of a patient after a myocardial intervention (MI), for example about 5-7 days after a MI. The endocardial gel injection may help to improve ischemic myocardial tissue recover with less fibrosis and increased angiogenesis and cardiac function. It can be appreciated that it may be technically challenging to reach an endocardial injection site, penetrate a needle at the injection site, and to make multiple injections (e.g., when desired/needed) while delivering the desired quantity of gel and while reducing/preventing back flow of the gel into the heart (e.g., the left ventricle).

Additionally, thermosensitive hydrogels (thermogels) may exhibit a phase transition near body temperature. For example, thermogels may be liquid at an environmental temperature and may solidify (e.g., undergo cross-linking) or increase in viscosity at the elevated physiological temperature in the body. Thermogels may aid in ischemic myocardial tissue recovery (e.g., inducing less fibrosis and/or increased angiogenesis and cardiac function). Yet, when administered via a long and/or narrow catheter through a blood vessel the thermogels may undergo warming (e.g., convective warming). Therefore, the thermogels may have reduced efficacy (e.g., may be prone to undergoing a phase change prior to reaching a target site (e.g., myocardial injection site) in the body.

Disclosed herein are systems for cooled endocardial injection, for example a cooled therapeutic gel after MI. The systems disclosed herein may overcome at least some of the technical challenges associated with endocardial injections. Some of the details of such systems are disclosed herein.

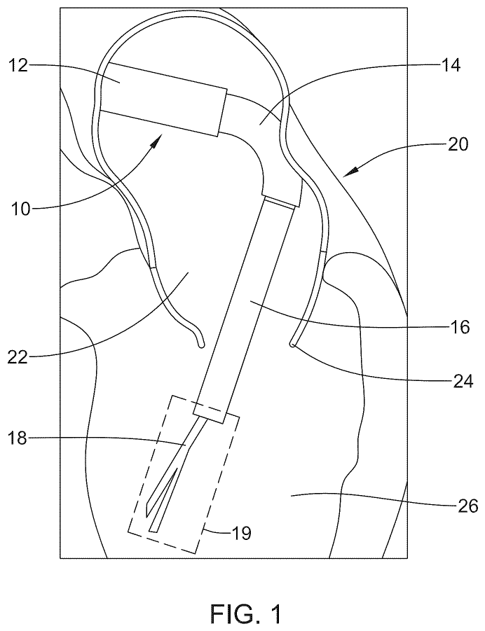

FIG. 1 illustrates an example system 10 for cooled endocardial injection (e.g., endocardial injection of cooled thermogels). The system 10 may include one or more catheters. For instance, the system may include one or more steerable catheters such as a first steerable catheter 12, a second steerable catheter 14, and a third steerable catheter 16 in a steerable catheter assembly. In this example, the steerable catheters 12, 14, 16 are depicted as being nested and/or otherwise arranged so that the outermost catheter (e.g., in this example the outermost catheter is the first steerable catheter 12) defines a lumen. The next inward or middle catheter (e.g., in this example the next inward catheter is the second steerable catheter 14) is disposed within the lumen. The middle catheter (e.g., in this example the second steerable catheter 14) defines a lumen and the innermost catheter (e.g., in this example the innermost catheter is the third steerable catheter 16) is disposed within the lumen. It can be appreciated that the numbering (e.g., first, second, and/or third steerable catheters 12, 14, 16) is not intended to be limiting and is not intended to be indicative of the quantity, arrangement or positioning of the steerable catheters 12, 14, 16. For example, in some instances the first steerable catheter 12 may be a middle catheter, an innermost catheter, or have any other suitable arrangement/position. The same is true of the other steerable catheters. Furthermore, so.me systems contemplated may include only a single steerable catheter, two or more steerable catheters, one or more “non-steerable” catheter intermixed with steerable catheters, and/or the like.

An injection catheter 18 may be disposed within one or more of the catheters 12, 14, 16. In this example, the injection catheter 18 may be disposed within the third steerable catheter 16 (e.g., within a lumen of third steerable catheter 16). In systems where less than three steerable catheters (e.g., two steerable catheters are utilized, and/or a combination of steerable and non-steerable catheters are utilized), the injection catheter 18 may be disposed within the lumen of one of the catheters of such a system. The injection catheter 18, in general, may be configured to deliver a therapeutic fluid to a target region. For example, the injection catheter 18 may be configured to deliver a therapeutic gel.

The system 10 can include a cooling circuit (not illustrated in FIG. 1), as described herein. The cooling circuit can be disposed along at least a portion of the steerable catheter. For example, the cooling circuit can be disposed at least partially within a wall of one or more of the catheters 12, 14, 16. For instance, the cooling circuit may be disposed in a wall of the middle catheter (e.g., in this example the second steerable catheter 14) and/or may be disposed in a wall of the innermost catheter (e.g., in this example the innermost catheter is the third steerable catheter 16). The cooling circuit can include a lumen configured to circulate cooling fluid therein. Therefore, the cooling circuit may extend along and be in contact with or may be proximate to the injection catheter and thereby may cool the injection catheter, as described herein.

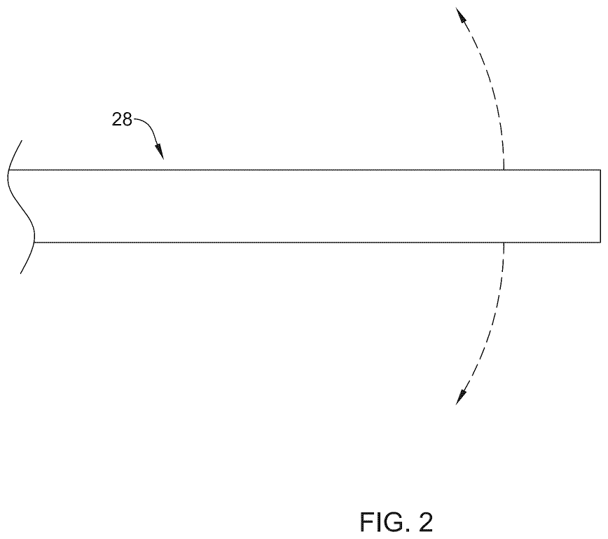

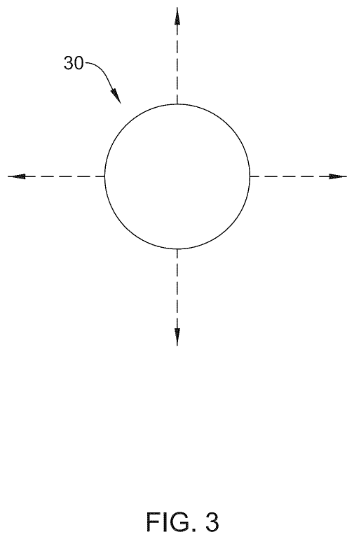

In use, the system 10 may be navigated into a heart 20. When doing so, the system may extend into a left atrium 22, through the mitral valve 24, and into the left ventricle 26. This may include advancing the system through the vena cava, performing a transseptal crossing through the atrial septum and into the left atrium 22. Other navigation methodologies are contemplated. As the names suggest, to help facilitate navigation, one or more of the catheters 12, 14, 16 may be steerable. This may include catheters steerable in at least one plane (e.g., yaw or pitch) and/or catheter steerable in at least two planes (e.g., yaw and pitch). For example, FIG. 2 schematically depicts a catheter 28, which may be representative of one or more of the steerable catheters 12, 14, 16, that is steerable in one plane. FIG. 3 schematically depicts a catheter 30, which may be representative of one or more of the catheters 12, 14, 16, that is steerable in two planes. FIGS. 2-3 are meant to depict that the steerable catheters 12, 14, 16 may be steerable in at least one plane or the steerable catheters 12, 14, 16 may be steerable in at least two planes. The structure of the steerable catheters 12, 14, 16 may vary to include pull wires, articulating structures/regions, and/or other structural features that help to allow the steerable catheters 12, 14, 16 to be navigated/steered.

It can be appreciated that navigating the system 10 to the heart 20 can include a trans-septal approach. Other approaches may be utilized including retrograde approaches where a trans-femoral and/or aortic approach is utilized. For example, the system 10 may be advanced through a femoral artery, through the aorta, and into the left ventricle.

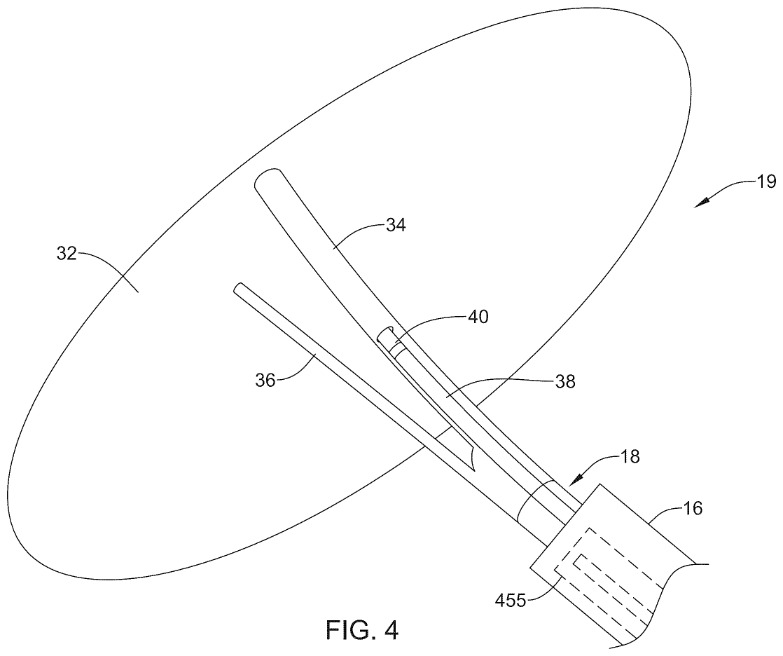

FIG. 4 is a plan view of a portion of an example system for cooled endocardial injection. Specifically, FIG. 4 illustrates an area 19 (as illustrated in FIG. 1) including the distal end region of the injection catheter 18 disposed at and/or engaged with a target region 32 and a distal end region of the steerable catheter 16.

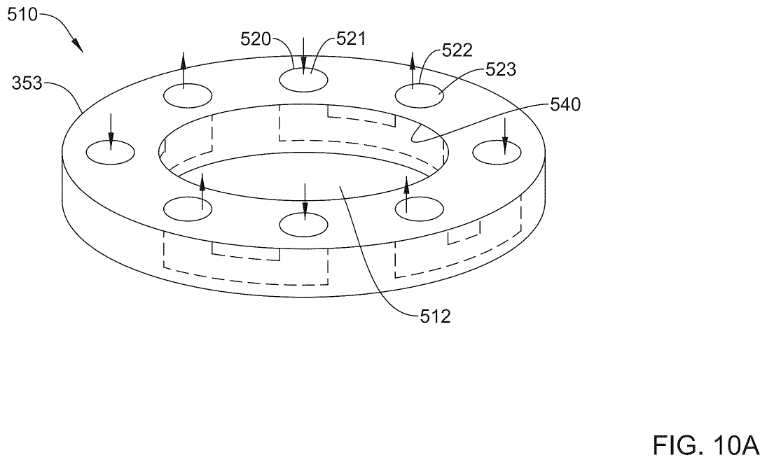

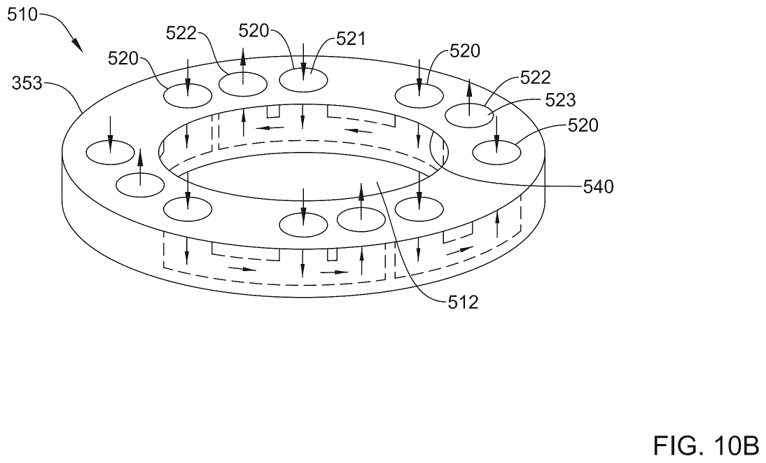

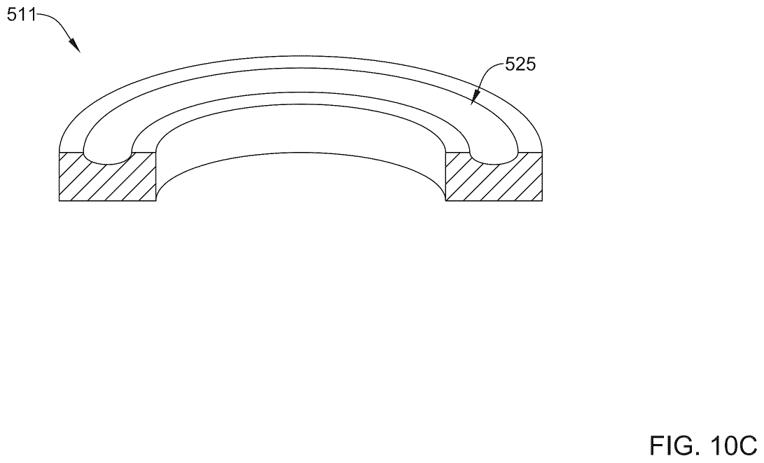

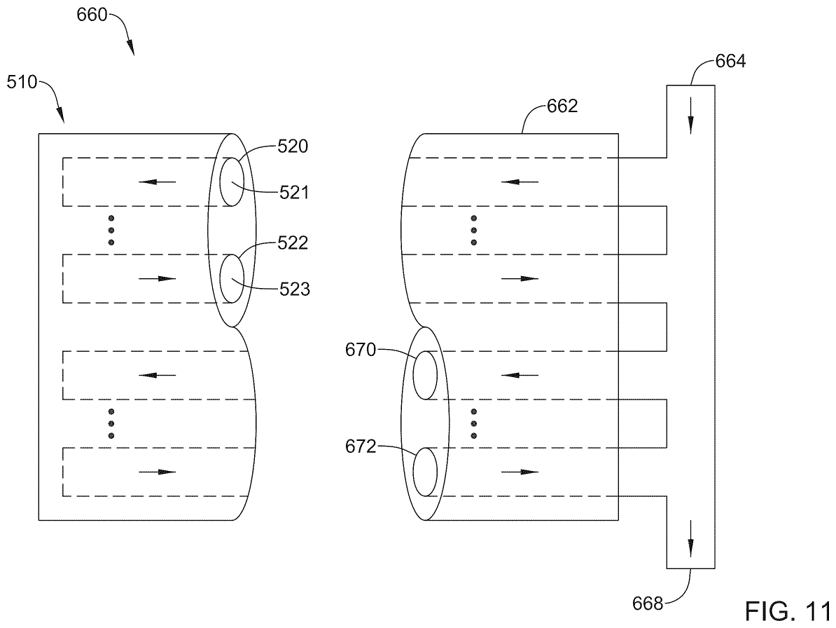

As illustrated in FIG. 4, the steerable catheter 16 can include a cooling circuit 455 disposed along at least a portion of the steerable catheter 16. As detailed herein, the cooling circuit 455 can be manifested at least in part as an elongate cooling circuit that includes a plurality of elongate lumen sections or a double helix cooling circuit that includes plurality of helical lumen sections. That is, in some instances the cooling circuit 455 may be manifested as an elongate cooling circuit (e.g., the elongate cooling circuit of FIG. 10A or FIG. 10B) with a plurality of elongate lumen sections that are disposed within a wall of a steerable catheter. Yet, in some instances the cooling circuit 455 may be manifested as a double helix cooling circuit with a plurality of helical lumen sections disposed in a wall of a steerable catheter such as a wall of the steerable catheter 16. While FIG. 4 illustrates the steerable catheter 16 as including the cooling circuit 455, in some instances the cooling circuit 455 may be present in a different steerable catheter such as the steerable catheter 14. That is, in some instances a distal end region of a steerable catheter in which the injection catheter 18 is nested may be a distal end region of the steerable catheter 14 or a distal end region of steerable catheter 12 such as in systems where less than three steerable catheters (e.g., two steerable catheters are utilized).

The cooling circuit 455 may be formed of a metal, a polymer, or a combination thereof. For instance, the cooling circuit 455 may be manifested as plurality of metal helical lumen sections. For example, the plurality of metal helical lumen sections may be stainless steel helical lumen sections, among other materials such as those as detailed herein. In some instances, the cooling circuit 455 may be manifested as an elongate polymer tube including a plurality of elongate lumen sections formed therein, among other materials such as those detailed herein.

It can be seen that a distal end region of the injection catheter 18 may include a first or imaging section 34 and a second or injection section 36. In some instances, the first section 34 may include or be termed an anchoring section. In some instances, the injection catheter 18 may be similar in form and function to the iNod™ Ultrasound Guided System, commercially available from Boston Scientific.

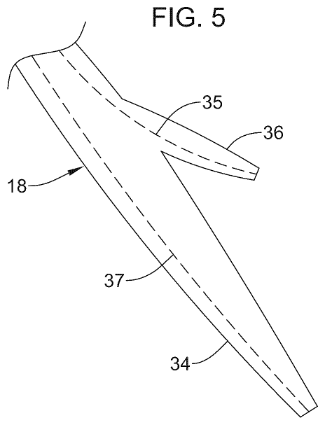

An imaging device 38 may be disposed within the injection catheter 18 and may be configured to extend along the imaging section 34. The imaging device 38 may include an imaging transducer 40 such as an ultrasound transducer. The imaging device 38 may be axially fixed within the imaging section 34. Alternatively, the imaging device 38 may be slidable within the imaging section 34. For example, the imaging device 38 may be disposed within a lumen (e.g., the lumen 37 schematically depicted in FIG. 5) formed in the injection catheter 18. In some instances, the imaging device 38 may extend all the way to the proximal end of the injection catheter 18 and be coupled to a handle or actuator (not shown) that may be used to control the position of the imaging device 38 within the injection catheter 18 (and/or the imaging section 34). Using axial translation and rotation of the imaging device 38, a three-dimensional cylindrical view can be built up, for example showing tissue/anatomy near/adjacent to the target region 32. This may allow the whole injection section 36 to be observed during tissue penetration and/or injection of a therapeutic fluid or gel. It can be appreciated that the imaging device 38 may be coupled to suitable hardware sufficient to energize and/or activate the imaging transducer 40. The imaging device 38 may also be coupled to suitable control and/or display hardware sufficient to display of anatomy imaged by the imaging device 38.

The injection section 36 may be used to pierce the target region 32. In some instances, the injection section 36 may take the form of or otherwise include a needle. In some of these instances, the injection section 36 may be shiftable and/or retractable relative to the injection catheter 18. For example, the injection section 36 may be retracted so that the end of the injection section 36 is disposed within the injection catheter (e.g., disposed within the lumen 35 schematically depicted in FIG. 5). Upon reaching the target region 32, the injection section 36 may be advanced into contact with the target region 32. When suitably positioned, a therapeutic fluid or gel may be injected into the target region 32 through the injection section 36. In some instances, the injection section 36 may be withdrawn while injecting the therapeutic fluid into the target region 32. It may be desirable to withdraw the needle at a rate slightly less than the injection fluid column velocity so that a desired injection pressure is maintained driving gel penetration into the parenchyma. The injection fluid column velocity may be determined by syringe/injection parameters.

Other control algorithms can be used optimize needle penetration depth. Such algorithms may utilize parameters such as myocardial depth (e.g., as measured by the imaging device 38), injection section 36 depth as measured by an external scale on the needle and/or an echogenic marker, the angle of the needle penetration in YAW and PITCH as measured by the imaging device 38. A suitable control and/or processing unit may be used in conjunction with the system 10 to facilitate visualization of injection section 36 depth/penetration and/or other suitable parameters.

It can be appreciated that the imaging device 38 can be used in conjunction with the injection section 36 in order to guide/navigate the system 10 in a manner that aid delivery of the therapeutic fluid to the desired location at/adjacent to the target region 32. For example, the imaging transducer 40 can be activated to image during navigation of the system 10 toward the target region 32, during actuation of the injection section 36 (e.g., when utilized), during engagement of the imaging section 34 and/or the injection section 36 with the target region 32, and/or during the infusion/injection of the cooled therapeutic fluid into the target region 32. This may include imaging across 360 degrees and/or stitching together multiple images in order to visualize injection boluses. In at least some instances, imaging may be able to discern the therapeutic fluid (e.g., discern between the therapeutic fluid and tissue) so that injection can be stopped if backflow is observed.

The therapeutic fluid may include a material such as a non-reversible thermosensitive gel. One example of a non-reversible thermosensitive gel that may be used is an ELR-SCAR gel. Such a substance may be a liquid at cooler temperatures (e.g., at about 17 degrees Celsius or lower) and may transform to a gel at higher temperatures including around body temperature (e.g., 37 degrees Celsius). Other materials contemplated include conformable embolic and/or sheer thinning materials such as OBSIOD™ Conformable Embolic material (commercially available from Boston Scientific), ORISE™ gel (commercially available from Boston Scientific), hyaluronic acid gel, reverse thermosensitive gels such as BACKSTOP gel (commercially available from Boston Scientific), combinations thereof, and/or the like. In some instances, the therapeutic fluid may include gas microbubbles, which may help to visualize the therapeutic fluid during/after injection.

FIG. 5, which is highly schematic in nature, illustrate some of the structures that may make up and/or otherwise be included with the injection catheter 18. For example, FIG. 5 depicts that the injection catheter 18 may include one or more lumens such as a lumen 37 in fluid communication with the imaging section 34 and/or a lumen 35 in fluid communication with the injection section 36. As indicated above, the lumen 37 may be used to house the imaging device 38. The lumen 35 may be used during the infusion/injection of the therapeutic fluid. In this example, the lumens 35, 37 are represented by dashed lines. The shape, path through the injection catheter 18, arrangement of the lumens 35, 37 within the injection catheter 18, combinations thereof, and/or the like can vary.

In cases where the therapeutic fluid used with the system 10 is ELR-SCAR, a cooling circuit in (e.g., in a wall of) one or more of the steerable catheters 12, 14, 16 may be used to help keep the therapeutic fluid at a desired temperature and/or state. It can be appreciated that cooling circuits may help to slow the gelling of the therapeutic fluid (e.g., an ELR-SCAR gel). For instance, the cooling circuit may be a closed cooling circuit configured to circulate (e.g., recirculate) a cooling fluid therethrough. Employing a closed cooling circuit may permit the use of various cooling fluids and/or operation temperatures (e.g., reduced operation temperatures) that may not be possible in open or semi-open cooling systems which may expose tissue directly to the cooling fluid. Accordingly, the cooling circuits herein may yield enhanced cooling (e.g., timely and/or effective) of the therapeutic fluid and/or a target site as compared to other approaches such as those that employ only open or semi-open cooling systems.

Additionally, in some embodiments, the passing of cooling fluid down the injection catheter 18 and/or one or more lumens in one or more of the steerable catheters 12, 14, 16 may help maintain the therapeutic fluid in a desired state (e.g., liquid). In such embodiments, flow through one or both of the lumens 35, 37 and/or through the lumens in one or more of the steerable catheters 12, 14, 16 may be set at about 1-50 milliliters (mL)/minute, or at about 5-30 mL/minute, or at about 5-10 mL/minutes, or at about 30 mL/minute. These flow rates may be for systems where flow is either through the end of the injection catheter 18 (e.g., an open flow system) or in a recirculation system within the injection catheter 18. These are just examples.

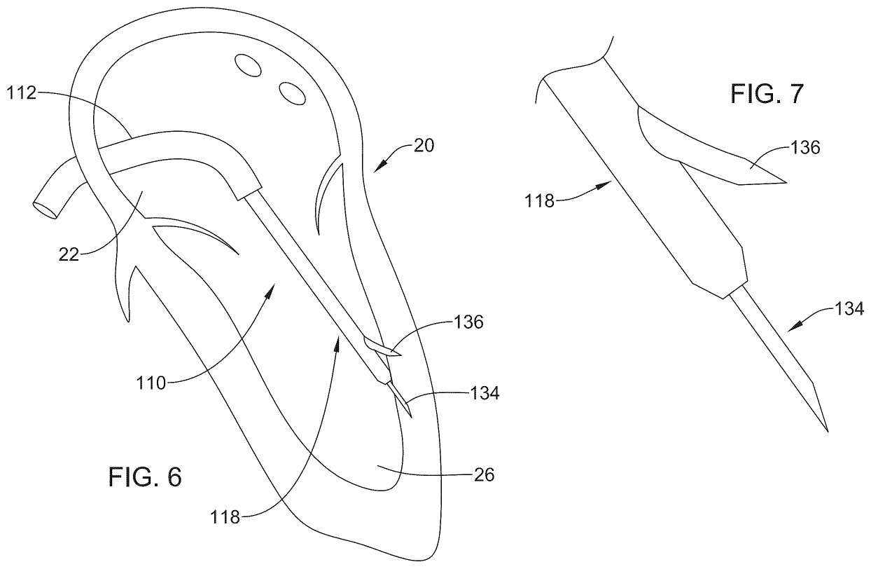

FIGS. 6-7 illustrate another example system 110 that may be similar in form and function to other systems disclosed herein. For example, the system 110 may include one or more steerable catheters, represented in FIG. 6 by reference number 112, and an injection catheter 118. The one or more steerable catheters 112 may include a cooling circuit, as described herein. As mentioned, the one more steerable catheters may be nested. In such instances, at least a steerable catheter of the one or more steerable catheters that is most proximate to the injection catheter may include a cooling circuit. For instance, the cooling circuit may be disposed in a wall of the steerable catheter that is most proximate axially to a wall of the injection catheter. Having the cooling circuit be disposed in a wall of the steerable catheter that is most proximate axially to a wall of the injection catheter can promote heat transfer between a cooling fluid in the cooling circuit and a therapeutic fluid and thereby mitigate a temperature increase of the therapeutic fluid prior to the therapeutic fluid reaching a target location in a body of a patient.

The injection catheter 118 may include first or stabilizing section 134 and a second or injection section 136. In this example, the stabilizing section 134 may take the form of a needle or tissue-engaging member that is configured to engage target tissues in order to facilitate stabilization and/or holding the position of the system 110. For example, the stabilizing section 134 may be used to pierce target tissue adjacent to a target location so that the injection section 136 can be used to inject a therapeutic fluid.

In some instances, the stabilizing section 134 may be retractable/advanceable. In other words, the stabilizing section 134 may be configured to shift between a first or delivery configuration (e.g., where the stabilizing section 134 is retracted into the injection catheter 118) and a second or stabilizing configuration (e.g., where the stabilizing section 134 is advanced out of the injection catheter 118 so as to allow/effect engagement with target tissue). The injection section 136, which may resemble other injection sections disclosed herein, may be similarly shiftable between a first or delivery configuration (e.g., where the injection section 136 is retracted into the injection catheter 118) and a second or injection configuration (e.g., where the injection section 136 is advanced out of the injection catheter 118 so as to allow/effect engagement with target tissue).

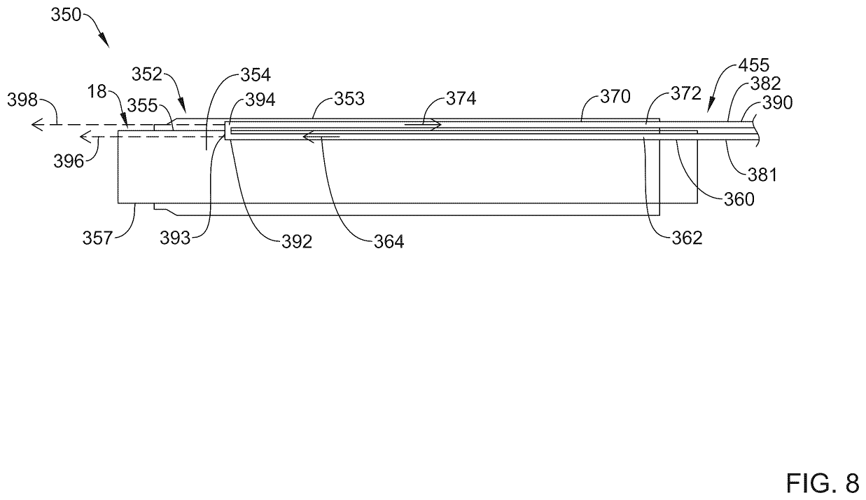

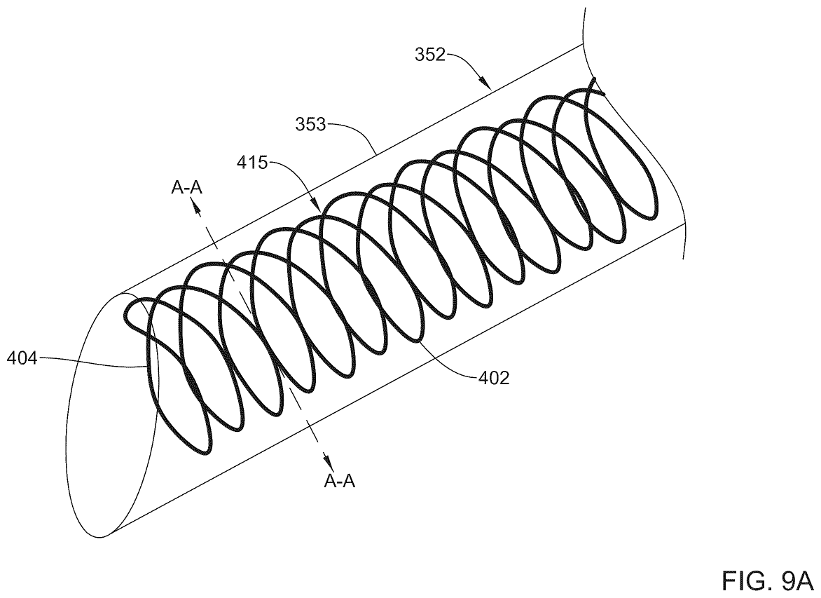

FIG. 8 is a schematic representation of a section view of a portion of an example system 350 for cooled endocardial injection. The system may be similar in form and function to other systems disclosed herein. Specifically, FIG. 8 is a schematic representation of a distal end region of a steerable catheter 352 (e.g., which may be similar in form and function to the steerable catheter 16) and a portion of the injection catheter 18.

The steerable catheter 352 can include a wall 353. The wall 353 can define a lumen 354 (e.g., a first lumen) of the steerable catheter 352. In some instances, an inner surface 355 of the wall 353 of the steerable catheter 352 can be in direct contact with an outer surface 357 of the injection catheter 18. Having the inner surface 355 of the wall 353 of the steerable catheter 352 be in direct contact with the outer surface 357 of the injection catheter 18 can promote heat transfer between a cooling fluid (not illustrated) in a cooling circuit 455 disposed in the steerable catheter 352 and a therapeutic fluid in a lumen of the injection catheter 18.

The cooling circuit 455 can be disposed along at least a portion of the steerable catheter 352. The cooling circuit 455 can include a fluid inlet 381 and a fluid outlet 382. The fluid inlet 381 and the fluid outlet 382 can each be configured to couple to a fluid circulation device (not illustrated). The fluid circulation device can be a pump or other component (e.g., a syringe) configured to circulate a cooling fluid through the cooling circuit 455. For instance, the fluid circulation device may be a pump that is coupled to both the fluid inlet 381 and the fluid outlet 382 and is configured to recirculate a cooling fluid through cooling circuit 455.

The cooling circuit 455 can include a plurality of lumen sections that extend along the steerable catheter. For example, the cooling circuit 455 can include a first lumen section 360 extending distally along the steerable catheter 352 and a second lumen section 370 extending proximally along the steerable catheter 352. That is, the first lumen section 360 and the second lumen section 370 can each extend between a proximal end 390 and a distal end 393 of the steerable catheter 352, where the first lumen section 360 includes the fluid inlet 381 and the second lumen section 370 includes the fluid outlet 382. The first lumen section 360 can include a lumen 362 therein. The second lumen section 370 can include a lumen 372 therein. Accordingly, the lumen 362 of the first lumen section 360 and the lumen 372 of the second lumen section 370 may together form a second lumen that extends between the fluid inlet 381 and the fluid outlet 382. That is, a distal end 392 of the first lumen section 360 and a distal end 394 of the second lumen section 370 can be fluidically coupled, as illustrated in FIG. 8. As such, the cooling circuit 455 can be configured to circulate cooling fluid distally along the lumen 362 of the first lumen section 360 and can return the cooling fluid proximally along the lumen 372 of the second lumen section 370. For instance, the cooling fluid (represented by element 364) can be circulated distally along the lumen 362 and the cooling fluid (represented by element 374) can be returned proximally along the lumen 372. Circulating the cooling fluid proximally along the lumen 362 and returning the fluid distally along the lumen 372 may promote heat transfer between the cooling fluid and a therapeutic fluid in the injection catheter. For instance, circulating the cooling fluid proximally along the lumen 362 and returning the fluid distally along the lumen 372 may promote cooling of the therapeutic fluid in the injection catheter such that cooled therapeutic fluid may be delivered to a target site in a body. Additionally, in some instances the approaches herein may employ closed cooling circuits that circulate the cooling fluid proximally along the lumen 362 and returning the fluid distally along the lumen 372. Such approaches thereby permit reuse of the cooling fluid, and yet mitigate any unwanted pressure changes (e.g., due to the introduction of cooling fluid at the target site, etc.) that may be typically associated with the use of open or partially open cooling systems. The cooling circuit could be partially open with the lumen of steerable catheter 352 acting as lumen 362 (i.e., distal flowing coolant) and lumen 372 allowing part of the coolant to move proximally. The remainder of the coolant exits into the blood via distal end of the steerable catheter 352. The amount of fluid flowing distally and proximally can be balanced so that only a desired minimal amount of coolant enters the bloodstream but at all times a positive pressure with respect to the blood pressure exists to prevent blood entering lumen 362, 372. The fluid in lumen 362, 372 flows in the space between the lumen and the outer surface of the injection catheter 357. The balance between input and output fluid is done by using syringes and controlling the actuation speed of the syringe plunger to control fluid flow. It is possible to have balancing so that all input exits as the output and no fluid enters the blood. The advantage of partial cooling as shown is the reduced space requirements enabling smaller catheter lumens overall.

In some embodiments, a longitudinal axis 396 of the first lumen section 360 and a longitudinal axis 398 of the second lumen section 370 can be substantially parallel. For instance, in some embodiments the longitudinal axis 396 of the first lumen section 360 and the longitudinal axis 398 of the second lumen section 370 can be parallel, as illustrated in FIG. 8. Having the longitudinal axis 396 of the first lumen section 360 and the longitudinal axis 398 of the second lumen section 370 be substantially parallel (e.g., parallel) can promote aspects herein such as providing a relatively short cooling fluid circulation path between the distal ends and the proximal ends of the lumen sections and thereby mitigate any unintended or undesired increase in temperature of the cooling fluid circulating in the lumen sections. However, in some instances the longitudinal axis 396 can be configured at non-zero angle relative to the longitudinal axis 398. For instance, the longitudinal axis 396 may be configured at non-zero angle relative to the longitudinal axis 398 during insertion and/or once inserted at a target site. The angle can be any angle that is less than or equal to about 180 degrees, less than or equal to about 90 degrees, or is less than or equal to about 45 degrees, etc.

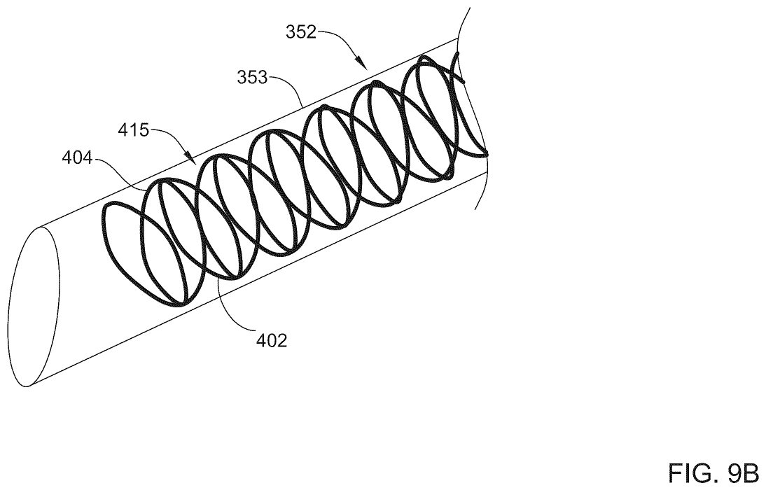

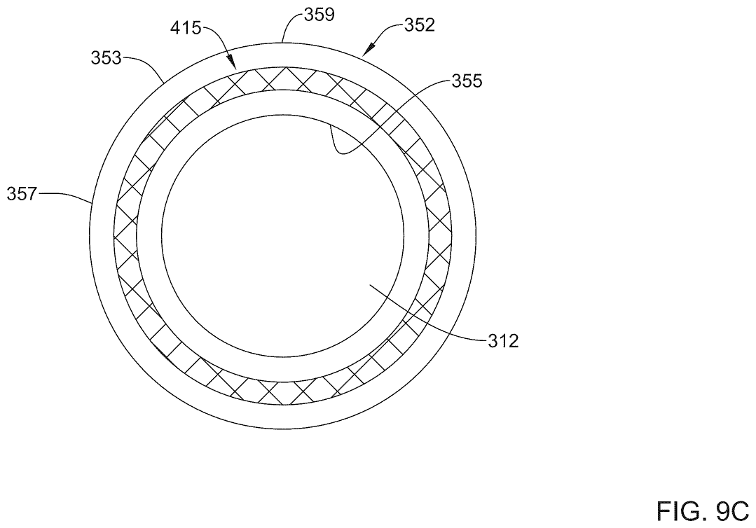

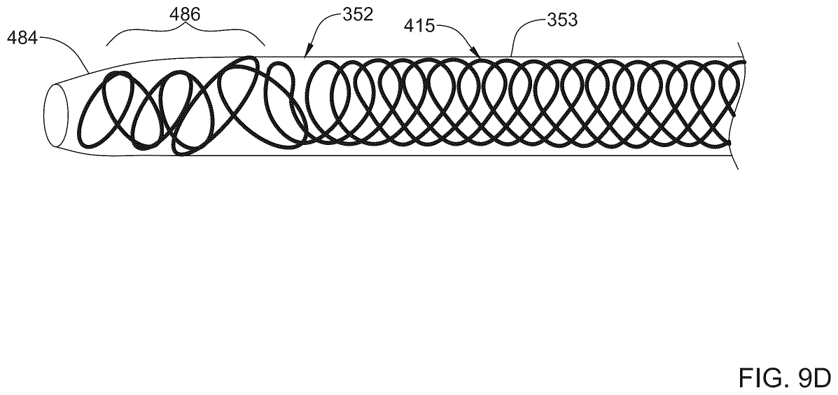

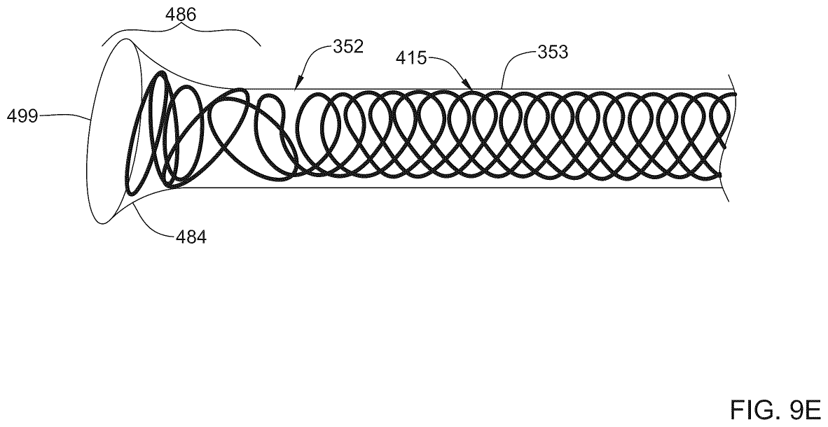

FIG. 9A is a view of an example of a double helix cooling circuit 415. As illustrated in FIG. 9A, a plurality of helical lumen sections may together form the double helix cooling circuit 415. For instance, the plurality of helical lumen sections can include two helical lumen sections such as a first helical lumen section 402 and a second helical lumen section 404 that form the double helix cooling circuit 415. The first helical lumen section 402 and the second helical lumen section 404 may be sections of a contiguous helical lumen having a portion thereof that extends in a distal direction (e.g., the first helical lumen section 402 may extend in the distal direction) and returns in a proximal direction (e.g., the second helical lumen section 404 may extend in the proximal direction) to form the double helix cooling circuit 415.

Adjacent helical elements in each of the first helical lumen section 402 and the second helical lumen section 404 may be spaced apart axially. For instance, adjacent helical elements in the first helical lumen section 402 can be spaced apart axially and adjacent helical elements in the second helical lumen section 404 can each be spaced apart axially. The axial spacing between adjacent helical elements may be uniform or may be non-uniform. For instance, adjacent helical elements may be configured at a given pitch such that successive helical elements along a longitudinal axis of the cooling circuit 415 have progressively smaller or larger spacing therebetween. In some instances, the helical elements in the first helical lumen section 402 and/or the second helical lumen section 404 may be configured at a pitch to have progressively smaller spacing in the distal direction (e.g., closer to a target sit). Having the adjacent helical elements be spaced apart axially along the steerable catheter can prompt aspects herein such as promoting uniform cooling of a therapeutic fluid in an injection catheter and/or enhancing steering of a steerable catheter 352 in which the double helix cooling circuit 415 is disposed.

As illustrated in FIG. 9A, each of the helical lumen sections can be the same size and shape. That is, the first helical lumen section 402 and a second helical lumen section 404 may have the same length (longitudinally) and/or same diameter (axially) that form the double helix cooling circuit 415. For instance, a length of each of the helical lumen section can be a value in a range from about 30 centimeters to about 180 centimeters or a value in a range from about 65 centimeters to about 140 centimeters, among other possibilities. In some instances, a diameter of each of the helical lumen sections can be a value in a range from about 1 millimeter to about 12 millimeters or a value in in a range from about 2 millimeters to about 10 millimeters, among other possibilities. Employing helical lumen sections with the same size and shape may promote aspects herein such as promoting uniform cooling and/or promoting ease of steering of the steerable catheter 352 in which the double helix cooling circuit 415 is disposed (e.g., disposed within a wall 353 of the steerable catheter 352).

However, in some embodiments, the helical lumen sections may have different lengths and/or different widths. For example, a first helical lumen section or cooling fluid delivery helical lumen section (e.g., extending distally along the steerable catheter and including a fluid inlet) that is in direct contact with a surface of the injection catheter (not shown in FIG. 9A) may have one or more relatively small helical elements (e.g., with respective inner lumen diameters that are relatively small), as compared to one or more helical elements in a second helical lumen section or cooling fluid return helical lumen section (e.g., extending proximally along the steerable catheter and including a fluid outlet). Employing helical lumens sections with helical elements that are different sizes may promote aspects herein. For instance, continuing with the above example employing a smaller first helical lumen section (e.g., extending distally along the steerable catheter and including a fluid inlet that is in direct contact with a surface of the injection catheter having relatively smaller helical sections) may increase an amount of surface area of the first helical lumen section that is in contact with the injection catheter and thereby promote heat transfer and yet, employing the second helical lumen section that is larger (e.g., has one or more helical elements with larger widths) may provide a return path for the cooling fluid and also may promote structural integrity and/or steerability of the a steerable catheter in which the double helix cooling circuit 415 is disposed.

The first helical lumen section 402 can be configured in a first helical direction and the second helical lumen section 404 can be configured in a second helical direction. For instance, as illustrated in FIG. 9A, the first helical direction and the second helical can be configured in the same helical direction (e.g., in a clockwise or counterclockwise manner). However, as illustrated in FIG. 9B, the first helical direction and the second helical can be configured in different helical directions. For instance, the first helical lumen section 402 can be configured in a first helical direction (e.g., a clockwise direction) and the second helical lumen section 404 can be configured in a second helical direction (e.g., a counterclockwise direction) that is different than the first helical direction, as illustrated in FIG. 9B.

FIG. 9C is a section view (along section line A-A in FIG. 9A) of an example of the cooling circuit in the form of the double helix cooling circuit 415. As illustrated in FIG. 9C, the double helix cooling circuit 415 can be disposed in a wall 353 of a steerable catheter 352. That is, the double helix cooling circuit 415 can be disposed between an inner surface 355 and an outer surface 359 of the wall 353 of the steerable catheter 352. As mentioned, an injection catheter (not shown in FIG. 9C) can be disposed in a lumen 312 in the steerable catheter 352. The injection catheter may include a third lumen and the injection catheter may be configured to deliver a cooled therapeutic fluid via the third lumen to a target site (e.g., endocardial tissue at the target site). Alternatively, as schematically depicted in FIGS. 9A-9B, the double helix cooling circuit 415 can be disposed along an inner wall surface of the steerable catheter 352.

In some embodiments, a portion or all of a cooling circuit may be formed of a shape memory material. For instance, at least a distal end of a cooling circuit may be formed of a shape memory material. For example, a distal end of a first helical lumen section and a distal end of a second helical lumen section of the double helix cooling circuit 415 may be formed of shape memory material. The shape memory material may be configured to shift between a first configuration and a second or expanded configuration. For instance, FIG. 9D is a view of an example of a steerable catheter including a shape memory material in a first configuration (e.g., an unexpanded configuration), while FIG. 9E is a view of an example of the steerable catheter including the shape memory material in a second configuration (e.g., an expanded configuration). In such embodiments, the shape memory material may transition from the first configuration to the second configuration due to a temperature increase of the shape memory material. For instance, the temperature increase may be at least partially attributable to insertion of the steerable catheter into a vessel of a body. Having the portion or all of a cooling circuit be formed of a shape memory material may promote aspects herein. For example, when the shape memory material is in the first configuration the distal tip 484 may fluidically seal a distal end of a lumen of the steerable catheter. For instance, as illustrated in FIG. 9D, a distal tip 484 of the steerable catheter may be configured to extend radially inward from the wall 353 of the steerable catheter when a shape memory material (represented as the material in a distal end 486) is in a first configuration (e.g., is not radially expanded).

However, when the shape memory material is in the second configuration the distal tip 484 may be radially expanded such that an opening 499 is formed and the lumen of the steerable catheter 352 is in fluid communication with an environment (e.g., a target area) surrounding the distal tip 484 of the steerable catheter. For instance, as illustrated in FIG. 9E, a distal tip 484 of the steerable catheter 352 may be configured to extend radially outward from the wall 353 of the steerable catheter 352 when a shape memory material (represented as the material in the distal end 486) is in a second configuration (e.g., is radially expanded). In such embodiments, a fluid may be passed through a lumen (e.g., of the steerable catheter and/or the injection catheter) when the distal tip 484 is in the second configuration. For instance, an additional cooling fluid (in addition to the cooled therapeutic fluid) may be passed through the lumen to further promote cooling of a target area, etc.

While FIG. 9D-9E illustrate the steerable catheter 352 as being formed continuously and including the distal tip 484, in some instances at least a portion of the steerable catheter 352 may be formed of a separate component that is coupled to a portion of the steerable catheter 352. For instance, the distal tip 484 may be a separate component (e.g., formed of a radially expandable material) that is coupled to a distal end of the steerable catheter, among other possibilities. In some instnaces, the distal end 486 may be formed as or include a structure that is integral with or continuous with the cooling circuit 415. Alternatively, the distal end 486 may include a separate/distinct shape memory structure that is coupled/secured to the cooling circuit 415. In some of these instances, the distal end 486 may have a helical arrangement similar to that of the cooling circuit. Other configurations/arrangements are contemplated.

FIG. 10A is a view of an example of an elongate cooling circuit 510. The elongate cooling circuit 510 may include a plurality of elongate lumen sections. In some embodiments, the quantity of elongate lumen sections can be an even quantity. Employing an even quantity of elongate lumen section may promote aspects herein such as having at least one fluid delivery lumen section that is fluidically coupled to at least one corresponding fluid return lumen section. For instance, the elongate cooling circuit 510 can include a total of eight elongate lumen sections. However, the elongate cooling circuit 510 can include a different (e.g., two, four, sixteen, etc.) quantity of elongate lumen sections. The plurality of elongate lumen section may include a first elongate lumen section 520 having a lumen 521 therein and a second elongate lumen section 522 having a lumen 523. The lumen 521 can be in fluid communication with a fluid inlet or fluid outlet in a proximal end of the steerable catheter and the lumen 523 can be in fluid communication with the other of the fluid inlet or the fluid outlet in the proximal end of the steerable catheter. In some instances, a distal end of the lumen 521 can be in fluid communication with a distal end of the lumen 523 to form closed cooling circuit that extends from a fluid inlet of the cooling circuit to the fluid outlet of the cooling circuit. As mentioned, the elongate cooling circuit 510 may be configured to be in fluid communication with a fluid circulation device to circulate cooling fluid through the cooling circuit.

In some instances, the plurality of elongate lumen section may be located within a wall of a steerable catheter and spaced radially from a center of a lumen 512 in the steerable catheter. For instance, the plurality of elongate lumen sections may be spaced radially around the wall 353 of the steerable catheter. For example, the plurality of elongate lumen sections may be spaced equally (e.g., the same distance from the center of the lumen 512) and may be uniformly spaced (relative to an adjacent elongate lumen section) equally around a circumference extending through a portion of the wall 353, as illustrated in FIG. 10A. Having the plurality of elongate lumen sections be equally and/or uniformly spaced (e.g., the same distance from the center of the lumen 512 and/or uniformly spaced from an adjacent elongate lumen section) may promote aspects herein such as uniform cooling of therapeutic fluid and/or case of steerability of a steerable catheter in which the elongate cooling circuit 510 is disposed.

In some instances, the first elongate lumen section 520 may be adjacent to the second elongate lumen section 522, as illustrated in FIG. 10A. Having the first elongate lumen section 520 be adjacent to the second elongate lumen section 522 may promote aspects herein such as forming a relatively short cooling fluid circulation path along the first elongate lumen section 520 and the second elongate lumen section 522. The relatively short cooling fluid circulation path may mitigate any unintended or undesired increase in temperature of the cooling fluid in the elongate lumen sections.

As illustrated in FIG. 10A an individual fluid delivery lumen such as the lumen 521 of the first elongate lumen section 520 may be paired with an individual fluid return lumen such as the lumen 523 of the second elongate lumen section 522. Stated differently, a 1:1 ratio of fluid delivery lumens to fluid return lumens can be employed. However, the ratio of fluid delivery lumens to fluid return lumens can be varied. For instance, a ratio of fluid deliver lumens to fluid return lumens can be 1:1, 2:1, 3:1, 4:1, 5:1, 6:1, among other possible ratios. For example, in some embodiments two fluid delivery lumens (e.g., two lumens 521 of the first elongate lumen sections 520) may each share a common fluid return lumen (e.g., the lumen 523 of the second elongate lumen section 522), as illustrated in FIG. 10B. In such embodiments, the fluid return lumen may have a larger diameter (e.g., a larger internal diameter) than the diameter of the fluid delivery lumens. Such a configuration may increase the distribution of the cooling fluid (e.g., among the fluid delivery lumens) and yet readily permit return of the cooling fluid via the fluid return lumens.

As mentioned, an injection catheter (not shown in FIG. 10A) can be disposed in the lumen 512 in the steerable catheter. The injection catheter may include a third lumen and being configured to deliver a cooled therapeutic fluid via the third lumen to endocardial tissue.

FIG. 10C is a view of an example of a portion of an elongate cooling circuit 511. The elongate cooling circuit 511 of FIG. 10C is similar to the elongate cooling circuits 510 in FIG. 10A and FIG. 10B, but with at least the difference that instead of a plurality of elongate lumen sections configured as elongate return lumen sections, an individual return lumen section 525 is employed. That is, one or more fluid delivery lumens (not illustrated) may be fluidically coupled to the individual return lumen section 525. For instance, an individual fluid delivery elongate lumen section (not illustrated) may be employed. The individual fluid delivery elongate lumen section may be an individual arcuate and may be configured at or approximate at a conjugate angle with the arcuate individual return lumen section 525. However, in some instances a plurality of fluid delivery elongate lumen sections may be employed with the individual return lumen section 525. In such instances, each of the plurality of fluid delivery elongate lumen sections may be fluidically coupled to the individual return lumen section 525. That is, each of the plurality of fluid delivery elongate lumen sections may deliver fluid distally along the injection catheter and may return the cooling fluid to the individual return lumen section 525 that extends proximally along the injection catheter.

FIG. 11 is a schematic view of a system 660 including an example cooling circuit and a manifold 662 (e.g., a cooling fluid manifold). The elongate lumen sections in an elongate cooling circuit such as the elongate cooling circuit 510 can be coupled to lumens in the manifold 662. Lumens such as the lumens 670 and 672 in the manifold 662 may be configured to supply cooling fluid to the elongate lumen sections in the elongate cooling circuit 510. For instance, the lumens 670 and 672 may be in fluid communication with fluid inlets and/or fluid outlets in the elongate lumen sections to supply fluid thereto.

In some instances, each elongate lumen section in the elongate cooling circuit 510 may have a corresponding lumen in the manifold 662. For instance, the first elongate lumen section 520 and the second elongate lumen section 522 may have individual lumens such as lumens 670, 672 in the manifold that correspond thereto. In such instances, a quantity of lumens in the manifold may be equal to or greater than a quantity of the elongate lumen sections in the elongate cooling circuit 510. However, in some instances, two or more of the elongate lumen sections in the elongate cooling circuit 510 may share a common cooling fluid supply and/or a common cooling fluid return in the manifold. As such, in some instances a quantity of lumens in the manifold 662 may be less than a quantity of lumens in the elongate cooling circuit 510.

The manifold 662 can include a fluid inlet 664 and a fluid outlet 668. Cooling fluid (e.g., chilled cooling fluid) may enter the fluid inlet 664, may circulate through one or more of the lumens in the manifold 662 and may circulate through elongate lumen sections of the elongate cooling circuit 510, and subsequently may exit via the fluid outlet 668. The fluid inlet 664 and the fluid outlet 668 may be coupled to various devices (not illustrated) that are configured to pressurize, exchange, filter, and/or cool the cooling fluid. The manifold 662 and/or the elongate cooling circuit 510 can include additional devices such as one-way valves (not illustrated) that control a direction and/or volume of flow of cooling fluid.

The materials that can be used for the various components of the system 10 (and/or other systems disclosed herein) may include those commonly associated with medical devices. For simplicity purposes, the following discussion makes reference to the catheter 12 and other components of the system 10. However, this is not intended to limit the devices and methods described herein, as the discussion may be applied to other similar tubular members and/or components of tubular members or devices disclosed herein.

The catheter 12 and/or other components of the system 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly (alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-clastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the system 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the system 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system 10 to achieve the same result.

In some embodiments, a degree of Magnetic Resonance Imaging (MRI) compatibility is imparted into the system 10. For example, the system 10, or portions thereof, may be made of a material that does not substantially distort the image and create substantial artifacts (e.g., gaps in the image). Certain ferromagnetic materials, for example, may not be suitable because they may create artifacts in an MRI image. The system 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Some examples of suitable cooling fluids include water, glycerol, nitrogen, carbon dioxide, or any combination thereof. For instance, the cooling fluid may be cooled water, cooled glycerol, liquid nitrogen, liquid carbon dioxide, or any combination thereof. As used herein, the term “cooled” refers to a cooling fluid that has a temperature which is below a physiological temperature of a patient. As used herein, the term “liquid” refers to a cooling fluid which has a definite volume but no fixed shape and that has a temperature below a physiological temperature of a patient.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A system for cooled endocardial injection, the system comprising:
a steerable catheter having a first lumen therein;
a cooling circuit disposed along at least a portion of the steerable catheter, the cooling circuit having a fluid inlet, a fluid outlet, a proximal end, and a distal end, the cooling circuit including:
a first lumen section extending distally along the steerable catheter and including the fluid inlet; and
a second lumen section extending proximally along the steerable catheter and including the fluid outlet; and
an injection catheter disposed in the first lumen, wherein the injection catheter is configured to deliver a cooled therapeutic fluid to endocardial tissue.

2. The system of claim 1, wherein a longitudinal axis of the first lumen section and a longitudinal axis of the second lumen section are parallel.

3. The system of claim 1, wherein the cooling circuit is disposed in a wall of the injection catheter.

4. The system of claim 1, wherein the cooling circuit is formed of a metal, a polymer, or a combination thereof.

5. The system of claim 1, wherein the cooling circuit is a closed cooling circuit.

6. The system of claim 5, wherein the closed cooling circuit includes a cooling fluid disposed therein, and wherein the cooling fluid comprises cooled water, cooled glycerol, liquid nitrogen, liquid carbon dioxide, or any combination thereof.

7. The system of claim 1, wherein:
the first lumen section is a first helical lumen section; and
the second lumen section is a second helical lumen section.

8. The system of claim 7, wherein adjacent helical elements in each of the first helical lumen section and the second helical lumen section are spaced apart axially.

9. The system of claim 8, wherein the first lumen section and the second lumen section together form a second lumen that extends between the fluid inlet and the fluid outlet.

10. The system of claim 8, wherein the first helical lumen section is configured in a first helical direction and the second helical lumen section is configured in a second helical direction.

11. The system of claim 7, wherein at least a distal end of the first helical lumen section and at least a distal end of the second helical lumen section are formed of shape memory material that is configured to move radially between an unexpanded configuration and an expanded configuration.

12. The system of claim 1, wherein:
the first lumen section is a first elongate lumen section; and
the second lumen section is a second elongate lumen section.

13. The system of claim 12, wherein the first elongate lumen section and the second elongate lumen section are included in a plurality of elongate lumen sections that are spaced axially along the steerable catheter.

14. The system of claim 13, wherein the plurality of elongate lumen sections are equally spaced axially along the steerable catheter.

15. The system of claim 1, wherein an inner surface of a wall of the steerable catheter is in direct contact with an outer surface of a wall of the injection catheter.

16. A system for cooled endocardial injection, the system comprising:
a steerable catheter assembly including a steerable catheter having wall with a first lumen therein;
a closed cooling circuit disposed within the wall of the steerable catheter, the closed cooling circuit having a fluid inlet and a fluid outlet each configured to couple to a fluid circulation device, a proximal end, a distal end, the closed cooling circuit including:
a first helical lumen section extending distally along the first lumen and including the fluid inlet; and
a second helical lumen section extending proximally along the first lumen and including fluid outlet, wherein the first helical lumen section and the second helical lumen section together define a second lumen that extends from the fluid inlet to the fluid outlet; and
an injection catheter disposed within the steerable catheter assembly, the injection catheter including a third lumen and being configured to deliver a cooled therapeutic fluid via the third lumen to endocardial tissue.

17. The system of claim 16, wherein the cooled therapeutic fluid is a thermogel.

18. A system for cooled endocardial injection, the system comprising:
a steerable catheter assembly including a steerable catheter having wall with a first lumen therein;
a closed cooling circuit disposed within the wall of the steerable catheter, the closed cooling circuit having a fluid inlet and a fluid outlet each configured to couple to a fluid circulation device, a proximal end, a distal end, the closed cooling circuit including:
a plurality of elongate lumen sections including a first elongate lumen section and a second elongate lumen section, wherein the first elongate lumen section and the second elongate lumen section together form a second lumen that extends between the fluid inlet and the fluid outlet; and
an injection catheter disposed within the steerable catheter assembly, the injection catheter including a third lumen and being configured to deliver a cooled therapeutic fluid via the third lumen to endocardial tissue.

19. The system of claim 18, wherein the first elongate lumen section is adjacent to the second elongate lumen section.

20. The system of claim 18, further comprising a manifold configured to fluidically couple a proximal end of the first elongate lumen section to the proximal end of the second elongate lumen section.
